(19) United States
(12) Patent Application Publication
(10) Pub. No.: US 2011/0224550 A1
(43) Pub. Date: Sep. 15, 2011

(54) ULTRASOUND DIAGNOSTIC SYSTEM AND METHOD FOR GENERATING STANDARD IMAGE DATA FOR THE ULTRASOUND DIAGNOSTIC SYSTEM

(75) Inventor: Dai Shinohara, Tokyo (JP)

(73) Assignee: HITACHI MEDICAL CORPORATION, Tokyo (JP)

(21) Appl. No.: 13/129,395

(22) PCT Filed: Nov. 10, 2009
(86) PCT No.: PCT/JP2009/069077

§ 371 Date: May 13, 2011

(30) Foreign Application Priority Data
Nov. 14, 2008 (JP) 2008-291707

(51) Int. Cl. A61B 8/14

(52) U.S. Cl. 600/443

(57) ABSTRACT

An ultrasound diagnostic system of the invention includes: an ultrasound probe that transmits and receives an ultrasound wave to and from an object; 3D position detection means configured to detect the position and inclination of a position sensor with respect to the object, the position sensor being mounted on the ultrasound probe; storage means configured to acquire and store 3D image data acquired by the ultrasound probe scanning on the body surface of the object and the position and inclination of the position sensor detected by the 3D position detection means; standard image data setting means configured to divide the 3D image data stored in the storage means into a plurality of slice image data and sets image position information and inclination information of a predetermined standard image data structure to the respective slice image data based on the position and inclination information of the position sensor detected by the 3D position detection means; and standard image data generation means configured to generate 3D standard image data by adding the image position information and inclination information set by the standard image data setting means to the respective slice image data.

### TECHNICAL FIELD

The invention relates to an ultrasound diagnostic system, and more particularly to a technique which enables the position information of image data to be used between the same or different ultrasound diagnostic systems or between an ultrasound diagnostic system and other modality imaging systems.

### BACKGROUND ART

Ultrasound diagnostic systems are widely used because of their capability to easily acquire real-time tomographic images of the internal features of an object. For example, since ultrasound diagnostic systems do not involve X-ray exposure unlike CT imaging systems, ultrasound diagnostic systems are ideal for diagnoses which lead to early detection of disease when performed periodically. When ultrasound diagnostic systems are used for such a purpose, it is preferable to make a diagnosis by comparing ultrasound images (still images) captured in the past and ultrasound images (still images) captured at the current time.

In this regard, Patent Document 1 proposes a technique in which the past volume data of an object such as a human body are acquired so as to be correlated with an object coordinate system, the coordinate information of tomographic planes (scanning planes) of ultrasound images captured at the current time is calculated in the object coordinate system, tomographic images having the same coordinate information as the calculated coordinate information of the tomographic planes are extracted from the volume data to reconstruct reference images, and the tomographic images and the reference images are displayed on a display monitor.

Moreover, when performing treatment on a lesion occurring in an internal organ such as the liver using an ultrasound diagnostic system, the following method of usage is known. That is, a treatment plan is established before treatment, a treated area is controlled during treatment, and the treated area is observed after treatment to see the effect of the treatment. In this case, it is useful to compare the ultrasound images with other modality images such as CT images which have a superior spatial resolution and a wider visual field than the ultrasound images. In observation during the preoperative, intraoperative, and postoperative treatments using the ultrasound diagnostic system, as described in Patent Document 1, it is helpful to display still images of other modality images corresponding to the ultrasound images of the treated area collated with other modality images such as MR images and PET images as well as the CT images and to compare the images with each other.

However, in the case of CT images, MR images, and the like, data elements that define the position information of each slice image on a 3D object coordinate system are standardized as a data structure of DICOM (Digital Imaging and Communication in Medicine) which is a NEMA (National Electrical Manufacturers Association) standard. According to this data structure, by setting DICOM data elements to image data such as CT images or MR images, parallel presentation of different modality images at the same slice positions, fusion of images, presentation or analysis of 3D positional relationship between images are made possible. That is, since 3D positional alignment of different modality images can be performed easily, various presentations and analyses are possible on various modality consoles, viewers, and the like in a hospital information system.

### CITATION LIST

### Patent Literature

[Patent Document 1] JP-A-2005-296436

### SUMMARY OF INVENTION

### Technical Problem

However, in the DICOM data structure that manages the attributes of ultrasound images, data elements that maintain the 3D position information of an image are not defined as standards. The reason therefor is because unlike other modality imaging systems, ultrasound diagnostic systems are easy to use and superior in their capability to display real-time ultrasound images on a monitor while capturing images and to capture images by freely changing the position and attitude of an ultrasound probe without fastening a patient who is an object to a bed or the like.

When comparing the past ultrasound images acquired with the ultrasound diagnostic system and ultrasound images acquired at the current time, it is not always easy to align the positions of the images since the object coordinate system and position information thereof are different. In addition, Patent Document 1 does not propose any specific method for achieving positional alignment between the object coordinate system of modality images captured by other imaging systems such as a CT system and the object coordinate system of ultrasound images acquired by an ultrasound system.

An object to be solved by the invention is to enable the position information of image data to be used between the same or different ultrasound diagnostic systems or between an ultrasound diagnostic system and other modality imaging systems.

### Solution to Problem

In order to attain the object, an ultrasound diagnostic system according to a first aspect of the invention includes: an ultrasound probe configured to transmit and receive an ultrasound wave to and from an object; 3D position detection means configured to detect the position and inclination of a position sensor with respect to the object, the position sensor being mounted on the ultrasound probe; storage means configured to acquire and store 3D image data acquired by the ultrasound probe scanning on the body surface of the object and the position and inclination of the position sensor detected by the 3D position detection means; standard image data setting means configured to divide the 3D image data stored in the storage means into a plurality of slice image data and set image position information and inclination information of a predetermined standard image data structure to the respective slice image data based on the position and inclination information of the position sensor detected by the 3D position detection means; and standard image data generation means configured to generate 3D standard image data by adding the image position information and inclination information set by the standard image data setting means to the respective slice image data.

A method for generating standard image data for the ultrasound diagnostic system according to the first aspect of the invention includes: a step wherein an ultrasound probe transmits and receives an ultrasound wave to and from an object; a step wherein 3D position detection means detects the position and inclination of a position sensor with respect to the object, the position sensor being mounted on the ultrasound probe; a step wherein a storage means acquires and stores 3D image data acquired by the ultrasound probe scanning on the body surface of the object and the position and inclination of the position sensor detected by the 3D position detection means; a step wherein standard image data setting means divides the 3D image data stored in the storage means into a plurality of slice image data and sets image position information and inclination information of a predetermined standard image data structure to the respective slice image data based on the position and inclination information of the position sensor detected by the 3D position detection means; and a step wherein standard image data generation means adds the image position information and inclination information set by the standard image data setting means to the respective slice image data to generate 3D standard image data.

As described above, according to the first aspect of the invention, the image position information and inclination information of a predetermined standard image data structure are set to the respective slice image data based on the position and inclination information of the position sensor detected by the 3D position detection means. Therefore, the image position information and inclination information of the respective ultrasound images captured by different ultrasound diagnostic systems can be represented by common data, and the position information of two image data can be used between different ultrasound diagnostic systems. By applying the standard image data structure of the invention to other modality imaging systems, the position information of image data can be used between an ultrasound diagnostic system and other modality imaging systems. In this case, a DICOM data structure can be used as the standard image data structure.

In this way, according to the first aspect of the invention, even when ultrasound images acquired in the past and ultrasound images acquired at the current time are captured by different ultrasound diagnostic systems, according to the 3D standard image data generated by the invention, since the image position information and the inclination information are defined by the same standards, by adjusting only the position of origin and the inclination of the images in the two object coordinate systems, for example, it is possible to easily align the positions of the images.

In the standard image data structure, the image position information may include the position of origin of an image and an arrangement spacing of slice images, and the coordinate of the origin of the image can be set at the center or the like of a pixel at the upper left corner of an image. Moreover, the inclination of the ultrasound probe can be represented as the inclination of an image, and can be represented by an inclination angle with respect to the respective axes (X-axis, Y-axis, and Z-axis) of an object coordinate system.

According to a second aspect of the invention, in the first aspect, the standard image data structure may further include a pixel spacing of the respective slice image data and the respective numbers of pixel rows and columns, and the standard image data setting means may calculate the intervoxel distance and the number of voxels based on the 3D image data to set the pixel spacing and the respective numbers of the pixel rows and columns of the standard image data structure of the respective slice image data. With this configuration, the position information of image data can be used between different ultrasound diagnostic systems or between an ultrasound diagnostic system and other modality imaging systems. Here, the pixel spacing is the distance between pixels that constitute a 2D slice image, and the respective numbers of pixel rows and columns are the respective numbers of pixels constituting the 2D slice image in the row and column directions.

According to a third aspect of the invention, in the first aspect, the ultrasound diagnostic system may further include coordinate conversion means configured to position the position sensor on an anatomically distinct portion of the object to adjust the position of origin of a position sensor coordinate system to the position of origin of an object coordinate system. With this configuration, since the standard image data structure can be defined in the object coordinate system, it possible to align the positions of two images more easily. As the anatomically distinct portion, at least one of the xiphisternum, the subcostal processes, and the hucklebone can be selected. In this case, by using plural anatomically distinct portions, it is possible to correlate the object coordinate system with the position sensor coordinate system with high accuracy.

Furthermore, in the first aspect, 2D standard images in 3D standard image data captured by other modality imaging systems may be displayed on a monitor as reference images, ultrasound images acquired by the ultrasound probe while adjusting the position and inclination of the position sensor may be displayed on the monitor, and the reference images and the ultrasound images may be compared on the monitor to adjust a coordinate system of the position sensor to an object coordinate system of the reference images so that the two images are made identical to each other.

With this configuration, through collation of ultrasound images and other modality images, the ultrasound images can be easily compared, for example, with CT images or the like which have a superior spatial resolution and a wider visual field. Particularly, during treatment planning or progress observation when performing ultrasound treatments, the ultrasound images can be compared with other modality images having a superior spatial resolution and a wider visual field. In this case, by storing the 3D standard image data using the data structure defined in DICOM as the standard image data structure, treatment planning or progress observation can be performed on a DICOM 3D display or the like.

According to a fourth aspect of the invention, in the first aspect, the ultrasound diagnostic system may further include body motion detection means configured to detect at least one body motion waveform of an electrocardiogram waveform and a respiratory waveform; the storage means may store time information corresponding to characteristic points of a body motion waveform detected by the body motion detection means while acquiring the 3D image data; the standard image data structure may include the time information of the body motion waveform; and the standard image data setting means may set the time information to the standard image data structure of the respective slice image data.

According to the fourth aspect of the invention, even when the ultrasound diagnostic system is not collated with other ultrasound diagnostic systems or other modality imaging systems, it is possible to realize effective use of a sole ultrasound diagnostic system using the standard image data structure according to the invention. For example, when making a diagnosis of a fetus, since the fetus moves in the body, it is not always important to detect the position in the object coordinate system. Moreover, since in most cases, there is no collation with other modality images, it is ideal to make the diagnosis such as observation of appearance using 3D ultrasound images which provide superior real-time images with no exposure. Moreover, 3D ultrasound images of bloodstream information enables obtaining information which may not be obtained in other modality images. In these diagnoses, the use of 3D ultrasound images having the standard image data structure enables detecting observation after examinations, changing the inclination, and the like. Furthermore, analysis processes such as 3D measurement can be performed later.

In addition, the invention enables applying a standard image data structure to an ultrasound diagnostic system which does not use 3D position detection means having a position sensor to realize effective use thereof. That is, an ultrasound diagnostic system according to a fifth aspect of the invention includes: an ultrasound probe that transmits and receives an ultrasound wave to and from an object; storage means configured to store 3D image data acquired by the ultrasound probe scanning in a direction perpendicular to a slicing cross-section of the object at a constant speed and generate and store the 3D position and inclination information of the ultrasound probe based on the scanning of the ultrasound probe; standard image data setting means configured to divide the 3D image data stored in the storage means into a plurality of slice image data and set image position information and inclination information of a predetermined standard image data structure to the respective slice image data based on the generated 3D position and inclination information of the ultrasound probe; and standard image data generation means configured to generate 3D standard image data by adding the image position information and inclination information set by the standard image data setting means to the respective slice image data.

A method for generating standard image data for the ultrasound diagnostic system according to the fifth aspect of the invention includes: a step wherein an ultrasound probe transmits and receives an ultrasound wave to and from an object; a step wherein a storage means stores 3D image data acquired by the ultrasound probe scanning in a direction perpendicular to a slicing cross-section of the object at a constant speed and generates and stores the 3D position and inclination information of the ultrasound probe based on the scanning of the ultrasound probe; a step wherein standard image data setting means divides the 3D image data stored in the storage means into a plurality of slice image data and sets image position information and inclination information of a predetermined standard image data structure to the respective slice image data based on the generated 3D position and inclination information of the ultrasound probe; and a step wherein standard image data generation means adds the image position information and inclination information set by the standard image data setting means to the respective slice image data to generate 3D standard image data.

According to the fifth aspect of the invention, it is possible to realize effective use of a sole ultrasound diagnostic system using the standard image data structure according to the invention. That is, depending on an ultrasound diagnostic area, there is a diagnostic area which has time-phase information, of which the shape changes from time to time in the same object, for example, as in a circulatory system as well as the heart or blood vessels. When capturing the images of such a diagnostic area, a method in which 3D images are acquired together with an electrocardiogram waveform or a heartbeat waveform associated with the change in the shape of the diagnostic area, still images synchronized with a particular time phase are acquired, and various diagnoses are performed is known. For example, a plurality of slice images corresponding to a particular time phase are acquired for a plurality of time phases while moving the slice position, and 3D behavior analysis of the heart, namely observation of the motion of valves, atria, and ventricles, and the volume of the atria and ventricles in each time phase, the change thereof, the amount of ejection, and the like can be performed using 3D images having a plurality of time phases. In this case, by generating the 3D standard image data using the standard image data structure, it is possible to easily make a diagnosis through comparison with the previous examinations.

A sixth aspect of the invention enables acquiring moving images by a sole ultrasound diagnostic system using the standard image data structure according to the invention to realize effective use thereof. That is, the ultrasound diagnostic system according to the sixth aspect of the invention includes: an ultrasound probe that transmits and receives an ultrasound wave to and from an object; 3D position detection means configured to detect the position and inclination of a position sensor with respect to the object, the position sensor being mounted on the ultrasound probe; storage means configured to acquire and store moving image data acquired by the ultrasound probe, time information of the moving image data, and the position and inclination of the position sensor detected by the 3D position detection means; standard image data setting means configured to set time information, image position information, and inclination information of a predetermined standard image data structure to the respective still image data of the moving image data stored in the storage means based on the time information and the position and inclination information of the position sensor detected by the 3D position detection means; and standard image data generation means configured to generate video standard image data by adding the time information, image position information, and inclination information set by the standard image data setting means to the respective still image data.

A method for generating standard image data for the ultrasound diagnostic system according to the sixth aspect of the invention includes: a step wherein an ultrasound probe transmits and receives an ultrasound wave to and from an object; a step wherein 3D position detection means detects the position and inclination of a position sensor with respect to the object, the position sensor being mounted on the ultrasound probe; a step wherein a storage means acquires and stores moving image data acquired by the ultrasound probe, time information of the moving image data, and the position and inclination of the position sensor detected by the 3D position detection means; a step wherein standard image data setting means sets time information, image position information, and inclination information of a predetermined standard image data structure to the respective still image data of the moving image data stored in the storage means based on the time information and the position and inclination information of the position sensor detected by the 3D position detection means; and a step wherein standard image data generation means adds the time information, image position information, and inclination information set by the standard image data setting means to the respective still image data to generate video standard image data.

According to the sixth aspect of the invention, when making a diagnosis of an area of which the shape is different between being in the resting state and being in a stressed state as in a circulatory system as well as the heart or blood vessels, the moving images in the resting state and the stressed state are acquired and stored, the change (motion) in the shape of each part of the diagnostic area is analyzed. In such a case, by generating the 3D standard image data of the diagnostic area using the standard image data structure of the invention and detecting the 3D position of an arbitrary cross-section, it is possible to easily make a diagnosis through comparison with the previous examinations.

### Advantageous Effects of Invention

According to the invention, the position information of image data can be used between different ultrasound diagnostic systems or between an ultrasound diagnostic system and other modality imaging systems.

### BRIEF DESCRIPTION OF DRAWINGS

FIG. 1 is a block configuration diagram of an ultrasound diagnostic system according to a first embodiment of the invention.

FIG. 2 is a configuration diagram of a collation system using the ultrasound diagnostic system of the first embodiment of the invention.

FIG. 3 is a conceptual diagram showing the processes of the first embodiment of the invention.

FIG. 4 is a flowchart showing a processing procedure of the first embodiment of the invention.

FIG. 5 is a diagram showing an example of a DICOM data structure.

FIG. 6 is a diagram illustrating the relationship between an arrangement of images in an object coordinate system and DICOM tags.

FIG. 7 is a diagram showing a representation example of position information of an ultrasound image in DICOM and an arrangement of images in the object coordinate system.

FIG. 8 is a flowchart showing a processing procedure of a second embodiment of the invention.

FIG. 9 is a conceptual diagram showing the processes of a third embodiment of the invention.

FIG. 10 is a flowchart showing a processing procedure of the third embodiment of the invention.

FIG. 11 is a conceptual diagram showing the processes of a fourth embodiment of the invention.

FIG. 12 is a flowchart showing a processing procedure of the fourth embodiment of the invention.

FIG. 13 is a conceptual diagram showing the processes of a fifth embodiment of the invention.

FIG. 14 is a flowchart showing a processing procedure of the fifth embodiment of the invention.

FIG. 15 is a conceptual diagram showing the processes of a sixth embodiment of the invention.

FIG. 16 is a flowchart showing a processing procedure of the sixth embodiment of the invention.

### DESCRIPTION OF EMBODIMENTS

Hereinafter, an ultrasound diagnostic system according to the invention will be described based on embodiments.

### First Embodiment

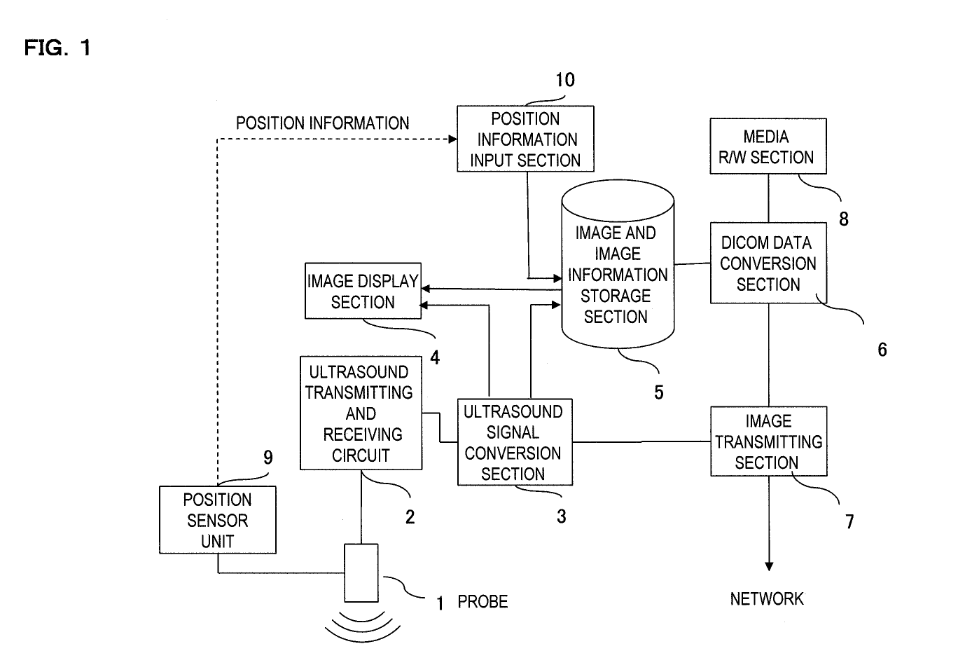

FIG. 1 shows a block configuration diagram of an ultrasound diagnostic system according to the first embodiment of the invention. As shown in FIG. 1, an ultrasound probe 1 has a well-known configuration, and is configured to transmit and receive an ultrasound wave to and from an object. A ultrasound transmitting and receiving circuit 2 drives the ultrasound probe 1 to transmit an ultrasound wave to an object and receive reflected echo signals generated from the object, performs predetermined signal reception processes to obtain RF data, and outputs the RF data to an ultrasound signal conversion section 3. The ultrasound signal conversion section 3 converts each RF frame data into 2D image data based on the input RF data and outputs the 2D image data to be displayed on an image display section 4 which is a monitor. Moreover, the ultrasound signal conversion section 3 stores a plurality of converted 2D image data in an image and image information storage section 5 which is a storage means as 3D image data.

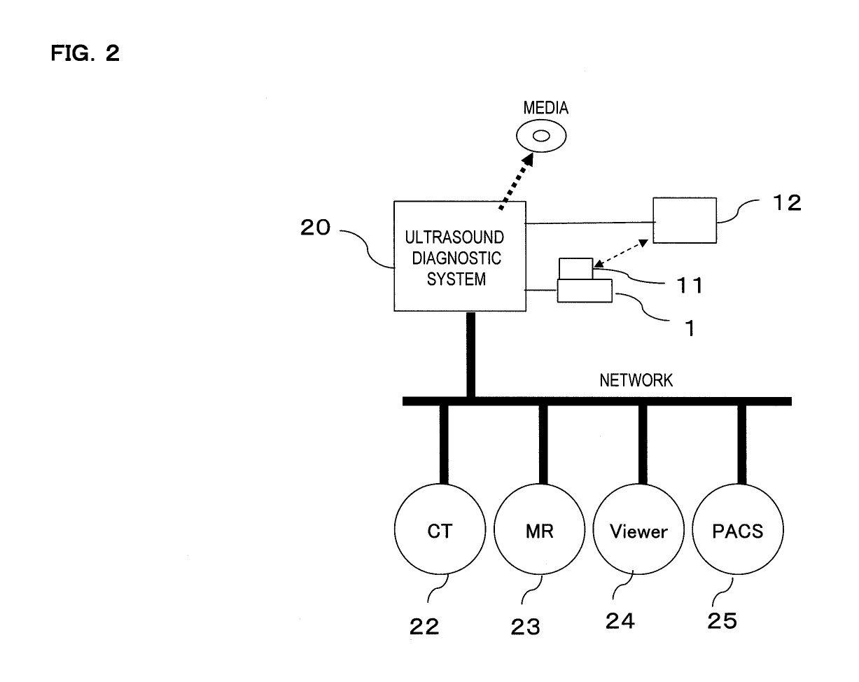

On the other hand, the ultrasound probe 1 is connected to a position sensor unit 9 serving as 3D position detection means. As shown in FIG. 2, the position sensor unit 9 includes a 3D position sensor 11 mounted on the ultrasound probe 1 and a transmitter 12 that forms a 3D magnetic field space, for example, around the object. The position information including the position and inclination of the position sensor 11 detected by the position sensor unit 9 is stored in the image and image information storage section 5 through a position information input section 10. The position information is stored in the image and image information storage section 5 so as to be correlated with respective RF frame data input from the ultrasound signal conversion section 3. In this way, in the image and image information storage section 5, 3D image data acquired when the ultrasound probe 1 scans on the body surface of the object and the position information of the position sensor 11 detected by the position sensor unit 9 are stored in a correlated manner.

A DICOM data conversion section 6 converts the 3D image data stored in the image and image information storage section 5 into well-known DICOM data which are one type of standard image data and stores the DICOM data again in the image and image information storage section 5. That is, the DICOM data conversion section 6 is configured to include a DICOM data setting means and a DICOM data generation means. The DICOM data setting means is configured to divide the 3D image data stored in the image and image information storage section 5 into a plurality of slice image data and set image position information and inclination information which are data elements of a predetermined DICOM data structure to the respective slice image data, based on the position information of the position sensor 11. The DICOM data generation means is configured to add the image position information and inclination information set to the respective slice image data to generate 3D standard image data and store the 3D standard image data in the image and image information storage section 5.

Moreover, as shown in FIGS. 1 and 2, the ultrasound signal conversion section 3 and the DICOM data conversion section 6 which constitute an ultrasound diagnostic system 20 are configured to be connected to a network through an image transmitting and receiving section 7 and transmit and receive image data to and from other modality imaging systems such as a CT 22 or an MR 23 or a DICOM server such as a Viewer 24 or a PACS 25, which are connected to the network.

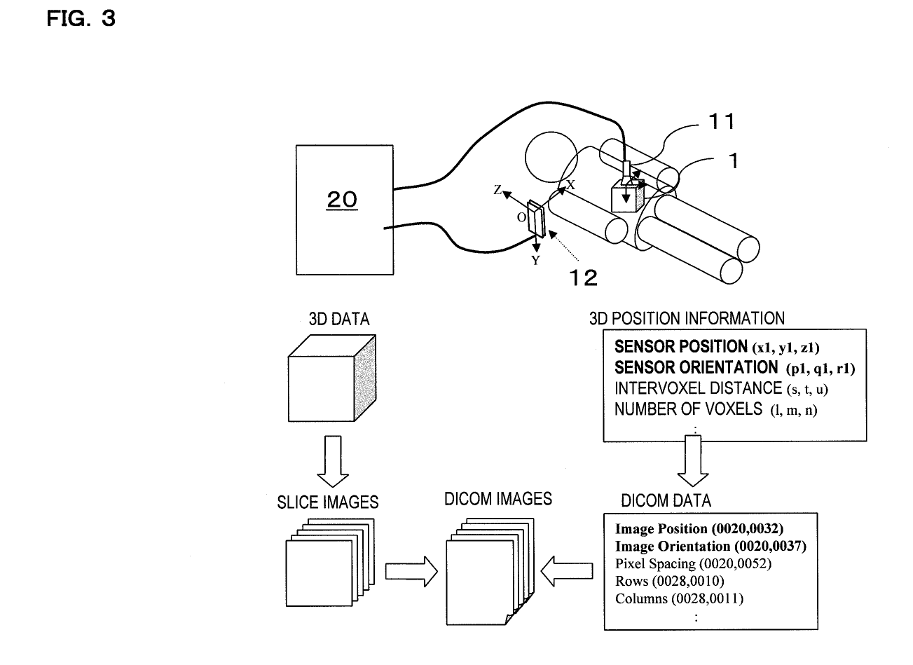

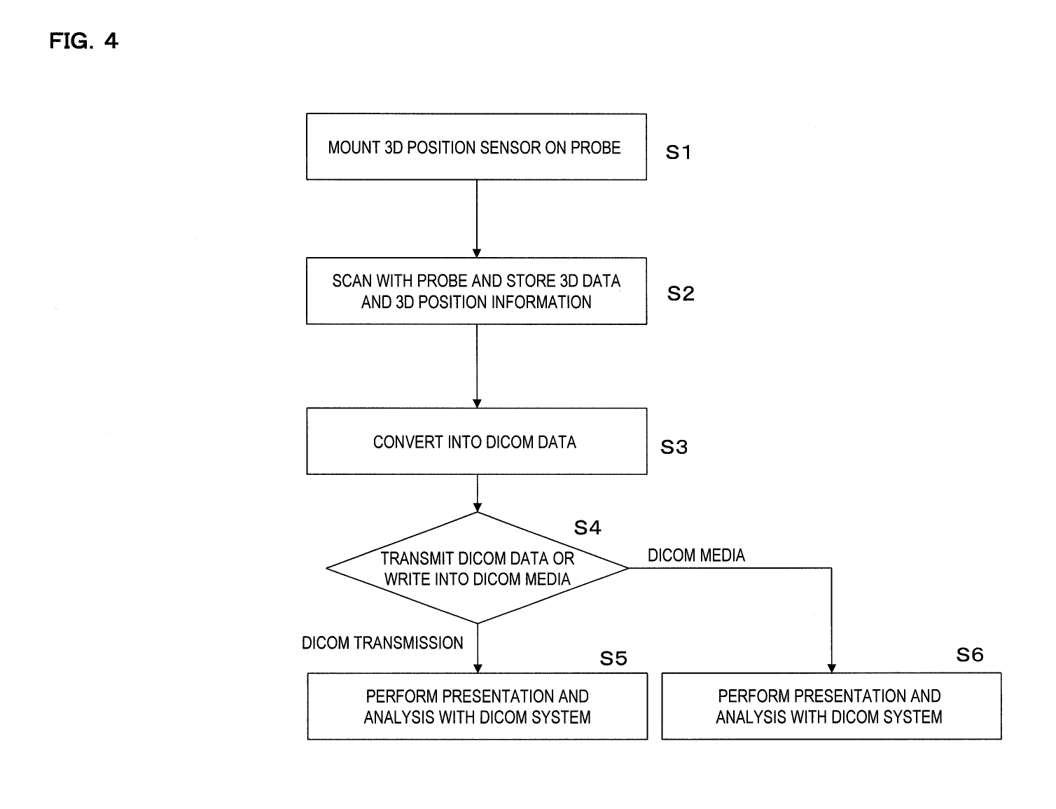

Here, a detailed configuration of the first embodiment will be described together with the operation thereof with reference to a conceptual diagram in FIG. 3 and a flowchart in FIG. 4. First, the 3D position sensor 11 is mounted on the ultrasound probe 1 (S1), and ultrasound 3D image data are acquired together with the 3D position information of the ultrasound probe 1 on a position sensor coordinate system and stored in the image and image information storage section 5 (S2). The 3D position information is made up of a sensor position (x1, y1, z1) and a sensor inclination (p1, q1, r1). Examples of the 3D position sensor include an optical position sensor and the like in addition to a magnetic position sensor as used in this embodiment, but the 3D position sensor is not limited to these sensors as long as they can detect the 3D position and inclination of the ultrasound probe 1. Moreover, the 3D image data may be acquired using a dedicated 3D ultrasound probe in addition to acquiring them by the ultrasound probe 1 scanning on the body surface. Furthermore, the format of the 3D image data is not particularly limited and may be voxel data, multi-slice data, and RAW (unprocessed) data. The image and image information storage section 5 may store images and image information in a memory, a database, a filing system, or a combination thereof.

Subsequently, the DICOM data conversion section 6 converts the DICOM data (S3). The converted DICOM data are transmitted to other modality imaging systems such as the CT 22 or the MR 23 or the DICOM server such as the Viewer 24 or the PACS 25 through the image transmitting section 7, or are written into DICOM media through a media R/W section 8 (S4). In the destination DICOM server, 3D presentation or 3D analysis is performed on the ultrasound DICOM images (S5). On the other hand, the DICOM data written into the DICOM media are read into a DICOM system and 3D presentation or 3D analysis is performed on the ultrasound DICOM images (S6).

Here, the detailed configuration and operation of the DICOM data conversion section 6 will be described. In the DICOM data conversion section 6, US Image Storage “Retired” or “New” is used as the type (SOP Class) of DICOM images. The US Image Storage does not consider whether the DICOM images are compressed or not.

Examples of the 3D position information of the position sensor 11 include an Image Position (0020, 0032), an Image Inclination (0020, 0037), and a Frame of Reference UID (0020, 0052), which are set as data elements corresponding to the DICOM data structure as will be described later.

Moreover, in the DICOM data element, a pixel spacing (0028, 0030), the number of pixel rows, Rows (0028, 0010), and the number of pixel columns, Columns (0028, 0011) are defined. Here, the intervoxel distance (s, t, u) and the number of voxels (l, m, n) are calculated based on the 3D image data, and the pixel spacing and the respective numbers of pixel rows and columns of the respective slice image data are set and converted into DICOM data. Moreover, the 3D image data stored in the image and image information storage section 5 are divided into a plurality of slice image data. Then, information corresponding to the data elements of the DICOM data structure set to the divided respective slice image data is set. In this way, the DICOM image data are generated. The generated 3D DICOM image data are stored in the image and image information storage section 5.

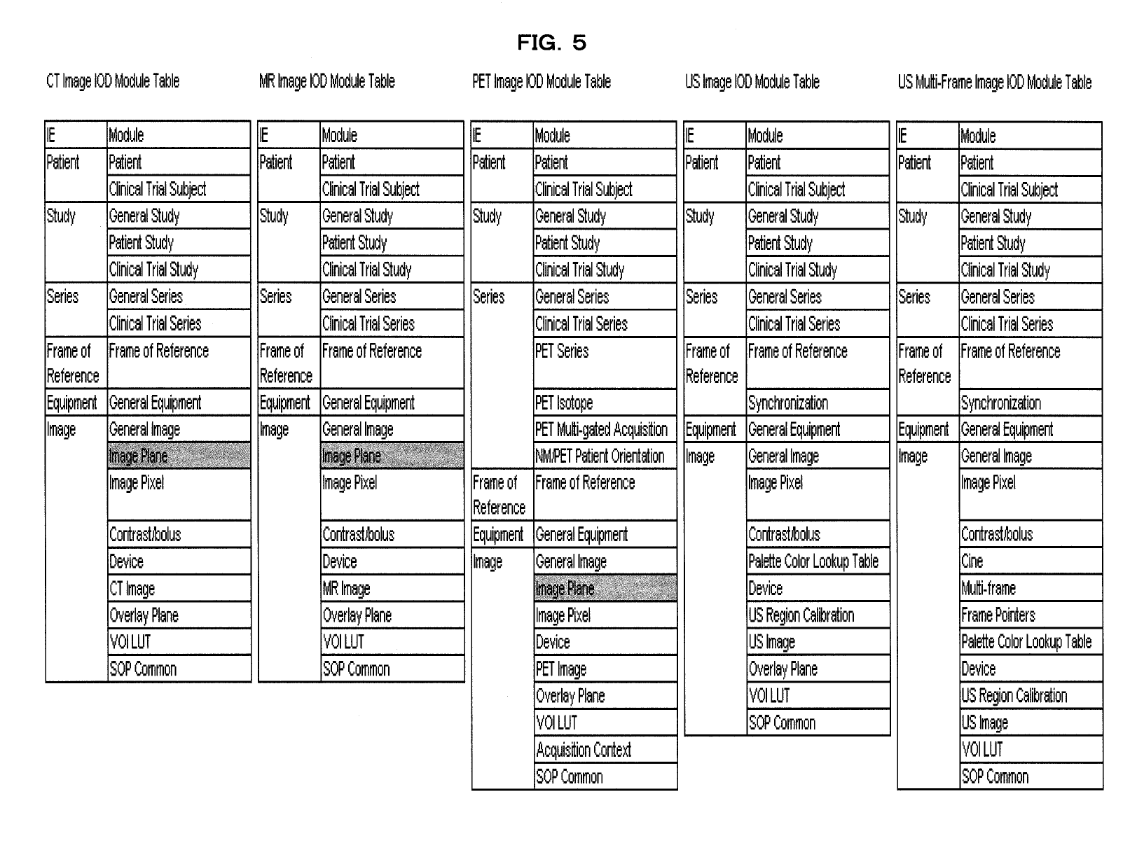

Here, the DICOM data structure and the data elements thereof will be described with reference to FIGS. 5 to 7. The DICOM data structure and the data elements thereof are described in the reference document, DICOM Part 3: Information Object Definitions (2007). As shown in FIG. 5, Image Plane modules including data elements that maintain the 3D position information of CT, MR, and PET images, and other images are defined in DICOM. Here, examples of the data elements maintaining the 3D position information include an Image Position (0020, 0032), an Image Inclination (0020, 0037), a Pixel Spacing (0028, 0030), and a Frame of Reference UID (0020, 0052) as described above.

These modality images generally have a table (bed) on which an object lies down and have features such that it is easy to substitute the amount of displacement of the table into the 3D position information. In contrast, as for ultrasound (US) images, as shown in FIG. 5, an Image Plane module including data elements that maintain the 3D position information is not defined. Therefore, the first embodiment proposes adding the 3D position information of US images to the DICOM data elements.

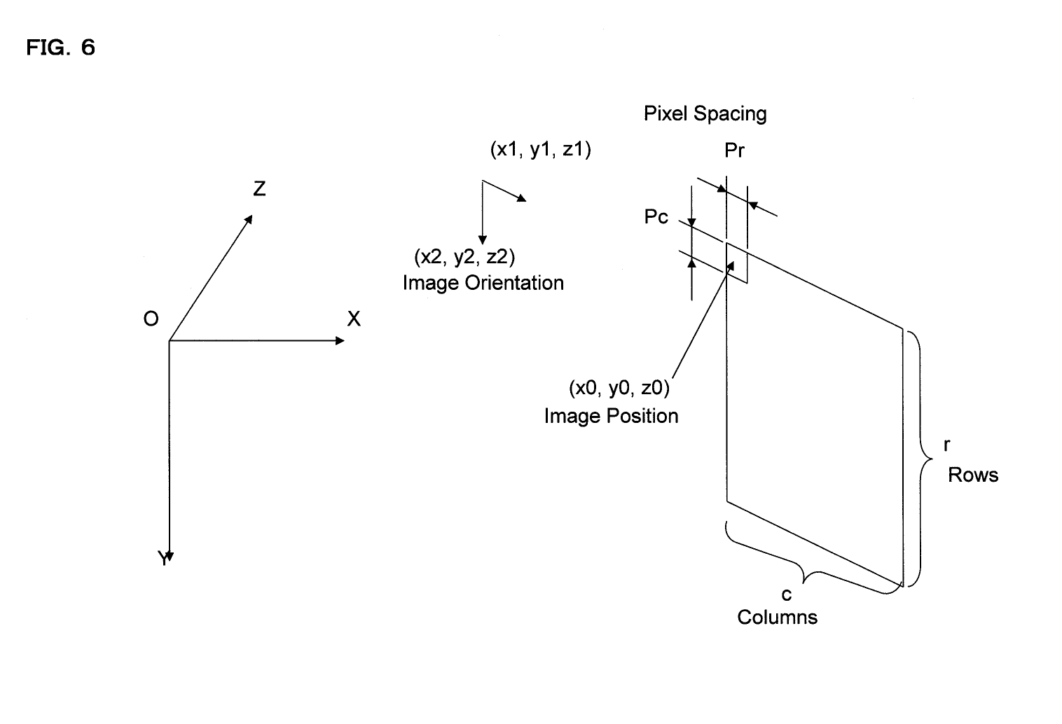

As shown in FIG. 6, the DICOM coordinate system is a right-handed system and is an object coordinate system which is based on an object. That is,

X direction: R (Right)→L (Left) direction

Y direction: A (Anterior)→P (Posterior) direction

Z direction: F (Foot)→H (Head) direction

Therefore, a 3D arrangement of an image in the object coordinate system is given by the following Tags.

Image position (Patient) (0020, 0032)
:(x0, y0, z0): [mm]: coordinate of a reference position, which is the central position of a pixel

Image inclination (Patient) (0020, 0037)
:(x1, y1, z1, x2, y2, z2): [−]: unit vectors in
   Raw and Column directions

Number of pixel rows, Rows, (0028, 0010)
:r[−]: Number of pixels in Column direction

Number of pixel columns, Columns, (0028, 0011)
:c[−]: Number of pixels in Row direction

Pixel spacing (0028, 0030)
:(Pr, Pc) [mm]: pixel spacing in Row and Column directions

Here, the respective numbers of pixel rows and columns are pixels at a reference position (in the example, the upper right corner) of an image.

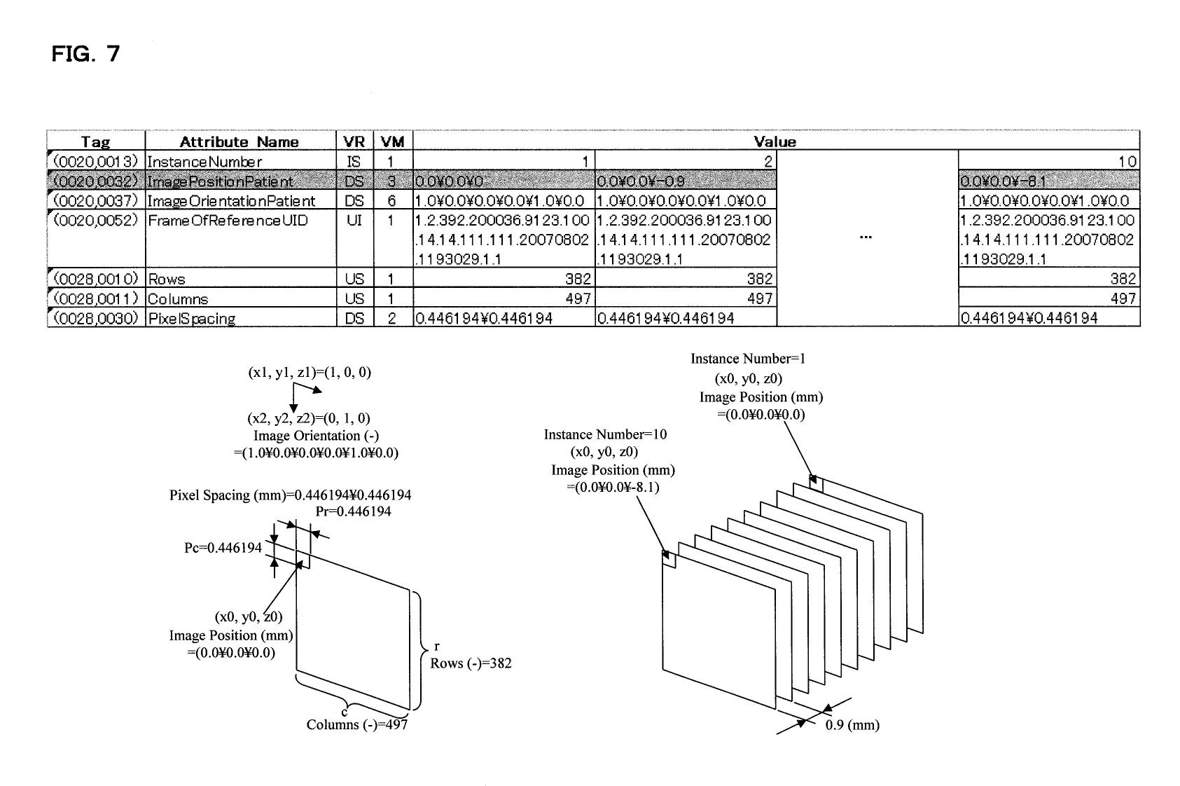

FIG. 7 shows an example of an expression in which 3D position information of ultrasound images is added to DICOM data elements. In the figure, it can be understood that the Value of the Image Position (Patient) (0020, 0032) is “0” for the first slice image, and the positions of the second and tenth images are changed from that position in the Z direction by an amount of “−0.9” mm and “−8.1” mm, respectively. Moreover, the Image Inclination (Patient) (0020, 0037) is the same. Furthermore, it can be understood that the number of pixel rows, Rows, is “382”, the number of pixel columns, Columns, is “497”, and the Pixel Spacing Pr and Pc are “0.4416194”.

By expressing the position information of ultrasound 3D image data based on the DICOM data of ultrasound images defined in such a way, the image position information and inclination information of the respective ultrasound images captured by different ultrasound diagnostic systems can be represented by common data, and the position information of two image data can be used between different ultrasound diagnostic systems. Moreover, in the present embodiment, since the ultrasound images can be expressed by DICOM data applied to other modality imaging systems, the position information of image data can be used between an ultrasound diagnostic system and other modality imaging systems.

The standard image data structure of the invention is not limited to the DICOM data structure but it is preferable to use the DICOM data structure as it is widely used.

Moreover, the ultrasound DICOM images generated by the present embodiment can be transmitted from the image transmitting and receiving section 7 shown in FIG. 1 to the DICOM server or can be written into media as DICOM files by the media R/W section 8. In this case, 3D presentation and 3D analysis of ultrasound DICOM images can be performed by the destination DICOM server or the DICOM system which reads the DICOM files through media. Here, the 3D presentation includes various rendering processes, MPR, and the like. Moreover, the 3D analysis includes 2D measurement of distances, angles, and the like on an arbitrary cross-section in addition to 3D measurement of volume or the like. Furthermore, the ultrasound diagnostic system 20 of the present embodiment may read ultrasound DICOM images and perform 3D presentation and 3D analysis on the ultrasound DICOM images.

As described above, according to the present embodiment, even when ultrasound images acquired in the past and ultrasound images acquired at the current time are captured by the same or different ultrasound diagnostic systems, according to the 3D standard image data generated by the invention, since the image position information and the inclination information are defined by the same standards, by adjusting only the position of origin and the inclination of the images in the two object coordinate systems, for example, it is possible to easily align the positions of the images.

Moreover, according to the present embodiment, since the pixel spacing of the slice image data and the respective numbers of pixel rows and columns can be set to the DICOM data, the position information of image data can be used between different ultrasound diagnostic systems or between an ultrasound diagnostic system and other modality imaging systems.

### Second Embodiment

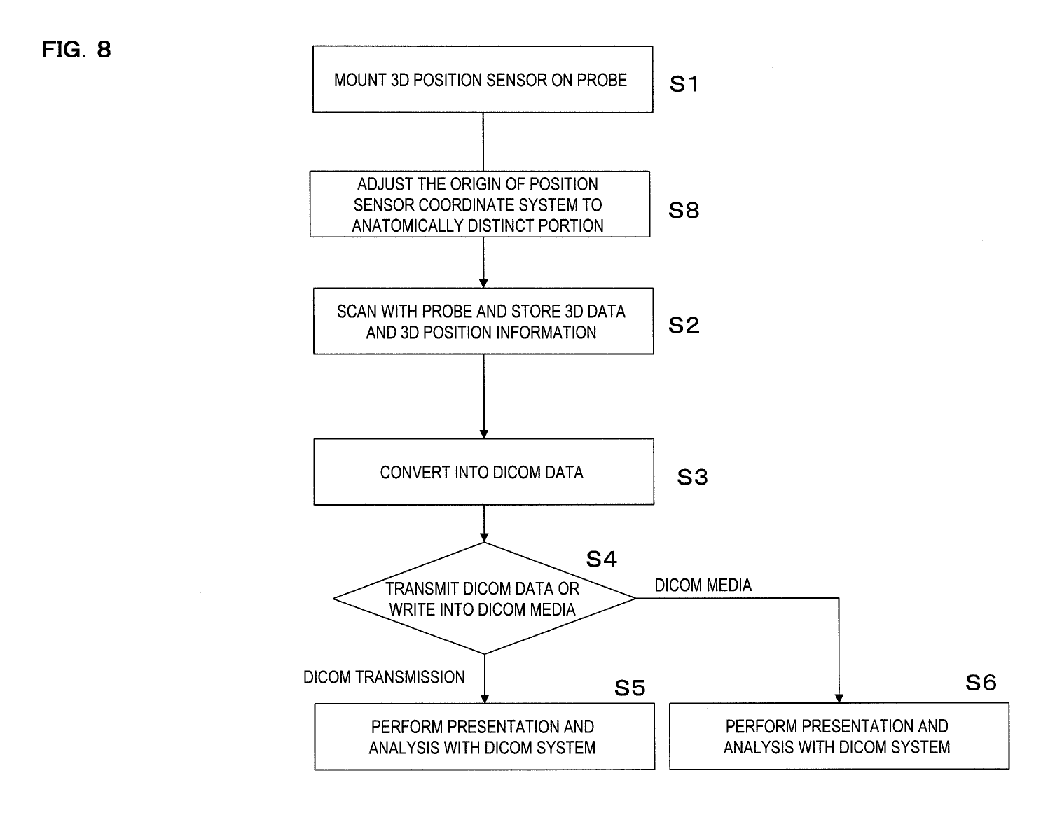

FIG. 8 shows a flowchart of a processing procedure in the second embodiment of the ultrasound diagnostic system of the invention. The present embodiment is different from the first embodiment in that it is provided with coordinate conversion means configured to adjust the position of origin of the coordinate system of the 3D position sensor 11 to the position of origin of an object coordinate system in which an anatomically distinct portion of an object is used as the origin. The other aspects are the same as those of the first embodiment, and description thereof will be omitted. As shown in FIG. 8, step S8 of adjusting the position of origin of the position sensor coordinate system to an anatomically distinct portion of an object is added at the end of step S1 in the flowchart of FIG. 4.

According to the present embodiment, since the position information detected by the position sensor 11 can be defined in the object coordinate system used by the DICOM image data, it is possible to align the positions of two images more easily. Moreover, for example, the ultrasound images obtained through several examinations can be compared easily. As the anatomically distinct portion, at least one of the xiphisternum, the subcostal processes, and the hucklebone can be selected. In this case, by using plural (for example, three) anatomically distinct portions, it is possible to make the inclination of the position sensor coordinate system aligned with respect to the object coordinate system and to acquire high-accuracy image position data.

### Third Embodiment

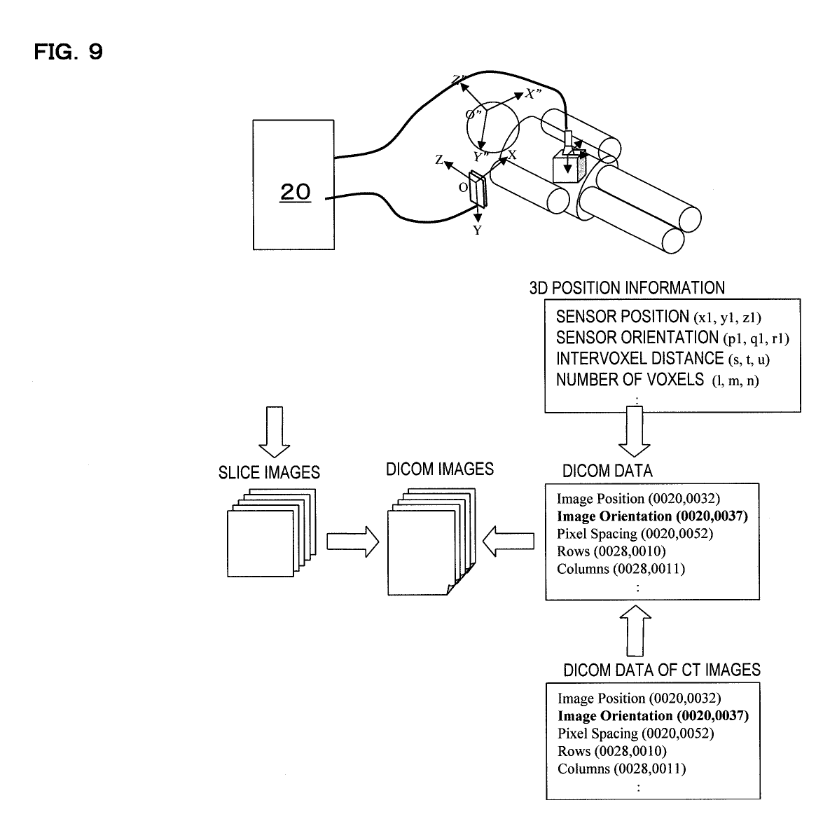

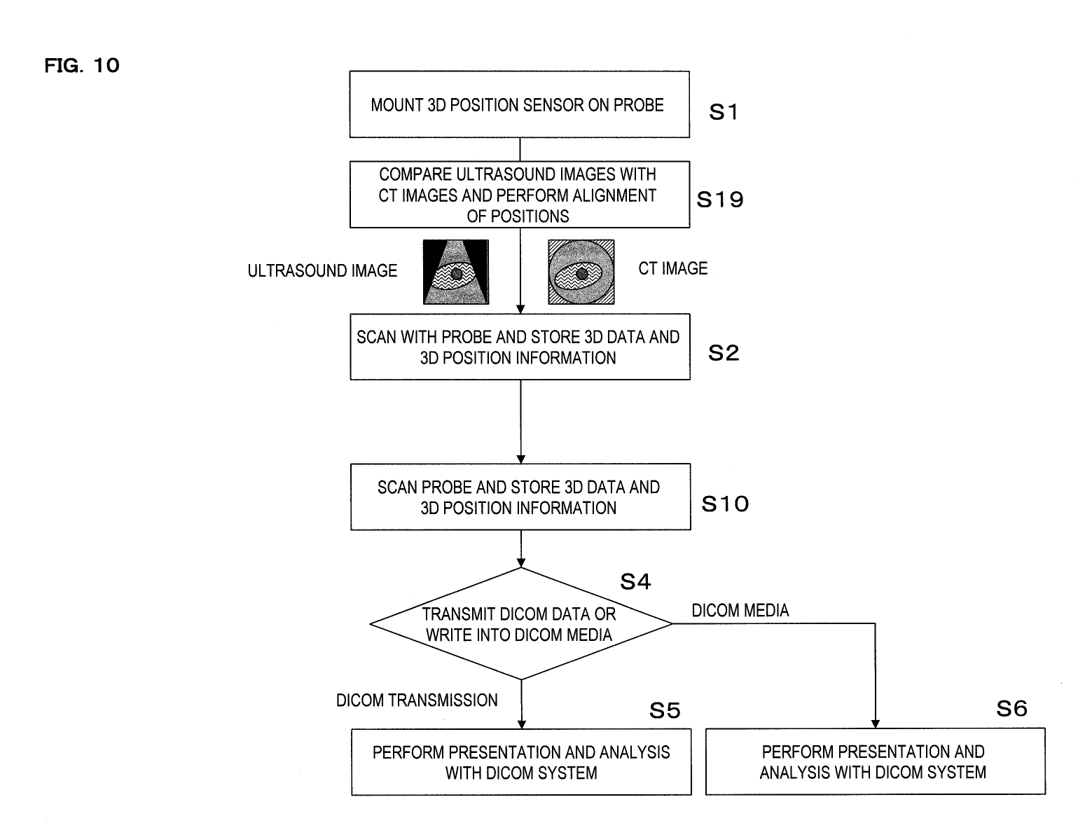

FIG. 9 shows a conceptual diagram of the third embodiment of the ultrasound diagnostic system of the invention, and FIG. 10 shows a flowchart of a processing procedure in the present embodiment. The present embodiment is different from the first and second embodiments in the following respects. That is, in the present embodiment, the position sensor coordinate system are displayed on a monitor with DICOM data captured by CT imaging systems which are other modality imaging systems as reference images, and ultrasound images are acquired while adjusting the position and inclination of the position sensor 11 and are displayed on a monitor. Then, the reference images and the ultrasound images are compared on the monitor to adjust the position sensor 11 to the object coordinate system of the reference images so that the two images are made identical to each other, whereby the position sensor coordinate system is made identical to the object coordinate system which is the coordinate system of the DICOM data of CT images.

That is, as shown in the flowchart of FIG. 10, step S8 of the second embodiment is replaced with step S9 of comparing real-time ultrasound images with reference images of DICOM data of CT images to make the position sensor coordinate system identical to the object coordinate system of CT images. Moreover, step S3 which involves conversion of DICOM data is replaced with step S10 in which DICOM data are converted in the object coordinate system of CT images.

According to the present embodiment, through collation of ultrasound images and other modality images, the ultrasound images can be easily compared, for example, with CT images or the like which have a superior spatial resolution and a wider visual field. Particularly, during treatment planning or progress observation when performing ultrasound treatments, the ultrasound images can be compared with other modality images having a superior spatial resolution and a wider visual field. In this case, by storing the DICOM 3D images using the data structure defined in DICOM as the standard image data structure, treatment planning or progress observation can be performed on a DICOM 3D display or the like.

The present embodiment may use MR images, ultrasound images, or the like as well as CT images. When setting the DICOM data elements of ultrasound images, the 3D position information is acquired from the DICOM data of CT images to obtain information on a CT object coordinate system. Moreover, the acquired 3D position information is converted in the CT object coordinate system using the position sensor coordinate system, and the DICOM data elements of the ultrasound images are set. In this way, the ultrasound images can be handled in the same object coordinate system as the referencing CT images.

### Fourth Embodiment

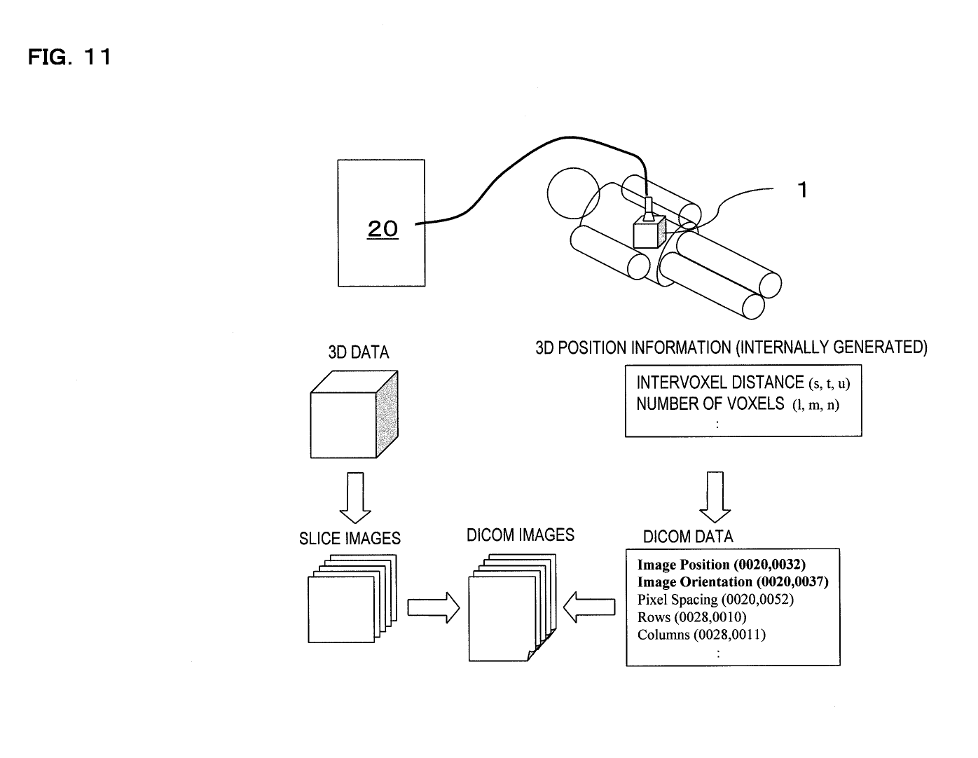

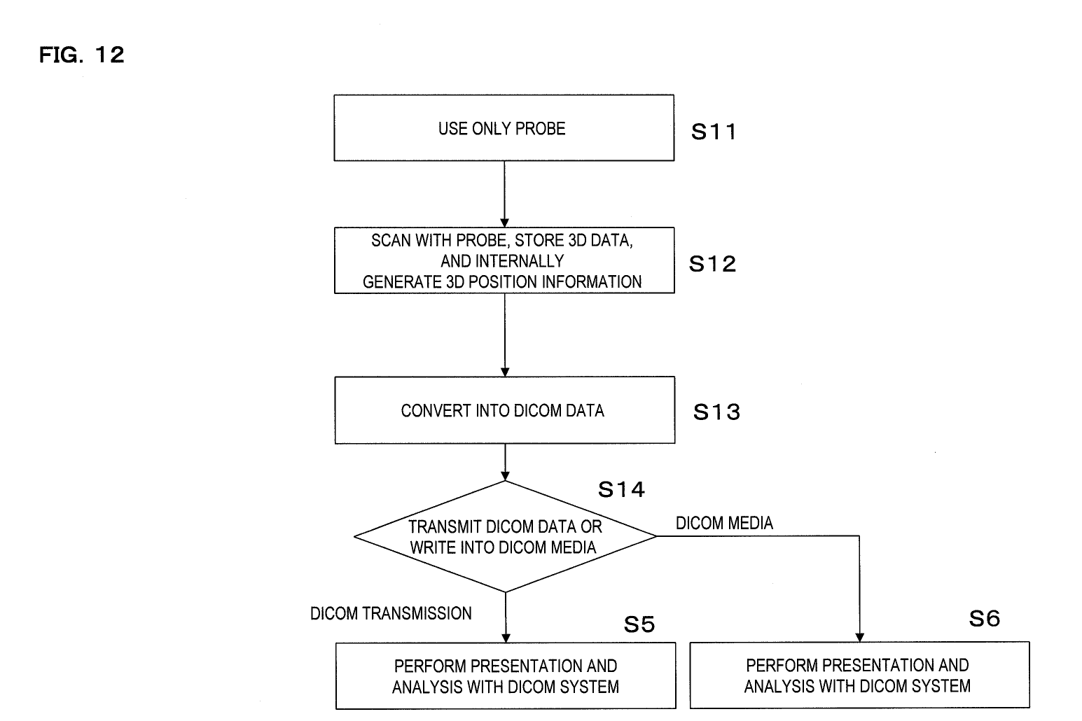

FIG. 11 shows a conceptual configuration diagram of the fourth embodiment of the ultrasound diagnostic system of the invention, and FIG. 12 shows a flowchart of a processing procedure of the present embodiment. The present embodiment is different from the other embodiments in that a standard image data structure is applied to an ultrasound diagnostic system which does not use position sensors to thereby realize effective utilization thereof.

That is, in the present embodiment, as shown in FIGS. 11 and 12, no position sensor is mounted on the ultrasound probe 1 (S11), 3D image data acquired by the ultrasound probe 1 scanning in a direction perpendicular to the slicing cross-section of the object at a predetermined constant speed are stored, and the 3D position information and inclination information of the ultrasound probe are internally generated based on the scanning conditions of the ultrasound probe 1 (S12). Subsequently, the DICOM data elements are set based on the internally generated 3D position information (S13).

In the case of the present embodiment, the setting of DICOM data elements in step S13 is different from the other embodiments in the following respects. First, the image position and the image inclination are set such that the row direction is X, the column direction is Y, and the probe scanning direction is Z using the center of a pixel at the upper left corner of an arbitrary slice position, for example, the first slice, as the position of origin. The other aspects are the same as those of the first embodiment or the like, and description thereof will be omitted.

For example, when making a diagnosis of a fetus, since the fetus moves in the body, although it is not always important to detect the position in the object coordinate system, it is ideal to make the diagnosis using ultrasound images which provide superior real-time images with no exposure. Particularly, presentation using 3D images is ideal for observation of the surface shape of a fetus, and observation of the appearance of the fetus is demanded to be provided to the family of the object as well as a physician. Moreover, 3D presentation of bloodstream information enables obtaining information which may not be obtained in other modalities. 3D analysis of a fetus is ideal for detecting the volume of a head part, the spine length, the femoral length, and the like. As for fetuses, a human body coordinate system and the relation with other modalities are not important. However, providing 3D images makes it easy to observe the appearance of a fetus and the bloodstream information. Moreover, providing 3D DICOM images enables observation after examinations, changing the inclination, and the like. Furthermore, analysis processes such as 3D measurement can be performed later.

### Fifth Embodiment

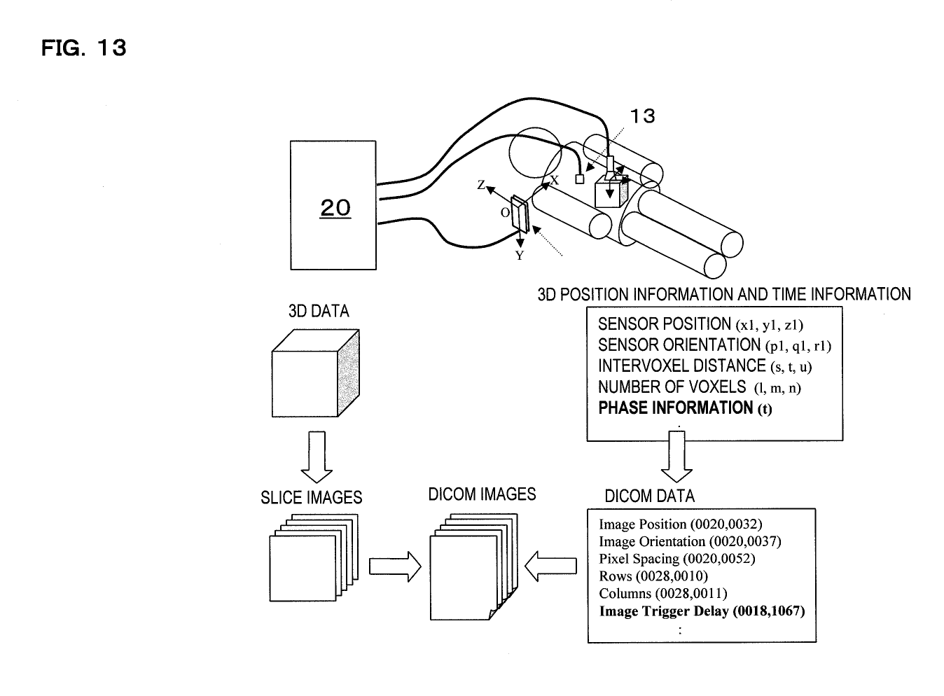

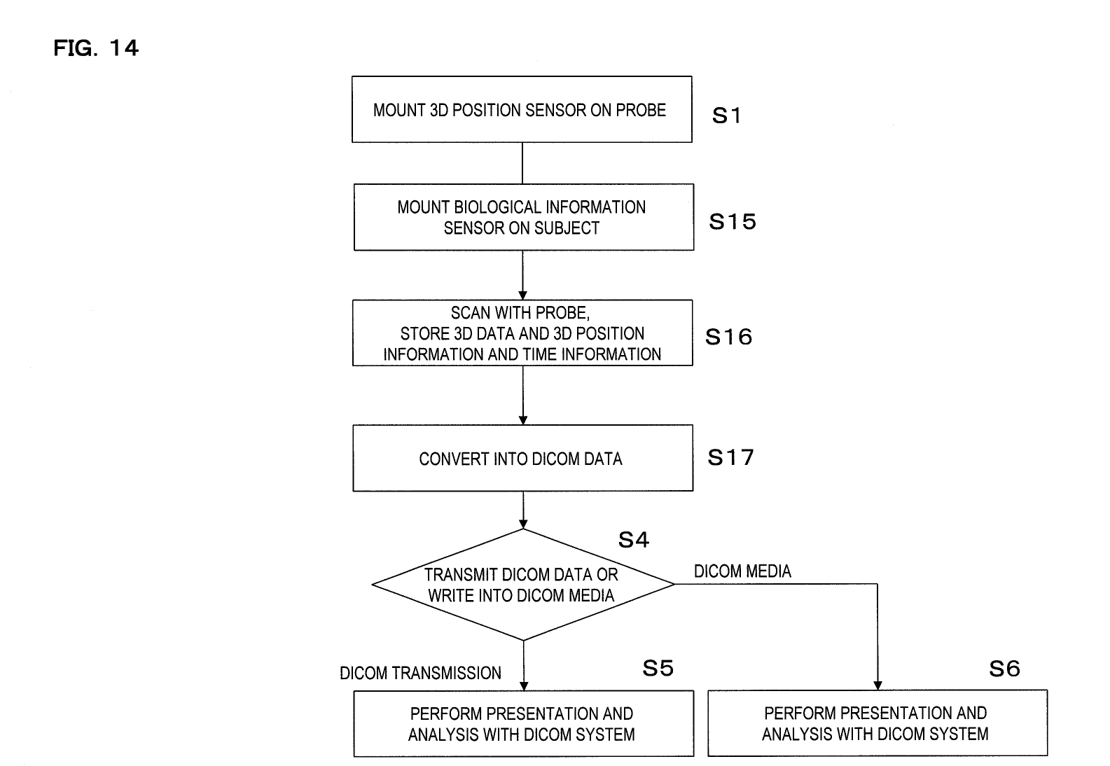

FIG. 13 shows a conceptual configuration diagram of the fifth embodiment of the ultrasound diagnostic system of the invention, and FIG. 14 shows a flowchart of a processing procedure of the present embodiment. The difference between the present embodiment and the other embodiments will be described. As shown in FIGS. 13 and 14, a biological information sensor 13 which is body motion detection means configured to detect at least one body motion waveform of an electrocardiogram waveform and a respiratory waveform is mounted on an object (S15). Subsequently, time information corresponding to characteristic points of the body motion waveform detected by the biological information sensor 13 is stored while acquiring 3D image data (S16). Moreover, the DICOM data conversion section 6 sets time information to the data elements of the time information of the body motion waveform, included in the DICOM data structure of the respective slice image data to convert the slice image data into DICOM data (S17). The other aspects are the same as those of the first embodiment, and description thereof will be omitted.

For example, in step S16, the slice position of an image is determined, and a delay time from an R wave is set while acquiring an electrocardiogram, for example. Then, images of respective time phases are acquired while moving the slice position of the image, whereby a plurality of slice images having a plurality of time phases are acquired. By using the 3D images having a plurality of time phases, 3D behavior analysis of the heart can be performed. As the 3D behavior analysis, the motion of valves, atria, and ventricles can be observed, and the volume of the atria and ventricles in each time phase, the change thereof, the amount of ejection, and the like can be measured. In this way, acquisition of a plurality of slice still images having time-phase information is effective for the 3D behavior analysis of the heart. As the time information, a time-phase delay from the R wave may be used for electrocardiogram synchronization, and a time-phase delay from the maximum expiration may be used for respiratory synchronization.

According to the present embodiment, it is possible to realize effective use of a sole ultrasound diagnostic system using the DICOM data structure. That is, depending on an ultrasound diagnostic area, there is a diagnostic area which has time-phase information, of which the shape changes from time to time in the same object, for example, as in a circulatory system such as the heart or blood vessels. When capturing the images of such a diagnostic area, a method in which 3D images are acquired together with an electrocardiogram waveform or a heartbeat waveform associated with the change in the shape of the diagnostic area, still images synchronized with a particular time phase are acquired, and various diagnoses are performed. For example, a plurality of slice images corresponding to a particular time phase are acquired for a plurality of time phases while moving the slice position, and 3D behavior analysis of the heart, namely observation of the motion of valves, atria, and ventricles, and the volume of the atria and ventricles in each time phase, the change thereof, the amount of ejection, and the like can be performed using 3D images having a plurality of time phases. In this case, by generating the 3D standard image data using the standard image data structure, it is possible to easily make a diagnosis through comparison with the previous examinations.

The DICOM data conversion section 6 divides the ultrasound 3D data into slice images and sets DICOM data elements including 3D position information and time information for each slice image. As the time information of the DICOM data elements, Image Trigger Delay (0018, 1067) is set, for example. The method of usage of the ultrasound DICOM images including the 3D position information and the time information is the same as that of the first embodiment. The DICOM system performs 4D presentation and 4D analysis of the ultrasound DICOM images.

The 4D presentation includes various rendering processes and the videos of MPR, and the like. The 4D analysis includes 2D measurement of the distances, angles, and the like in an arbitrary cross-section for each time phase in addition to 3D measurement of volume or the like for each time phase. Moreover, the ultrasound diagnostic system 20 may read ultrasound DICOM images and perform 4D presentation and 4D analysis on the ultrasound DICOM images.

### Sixth Embodiment

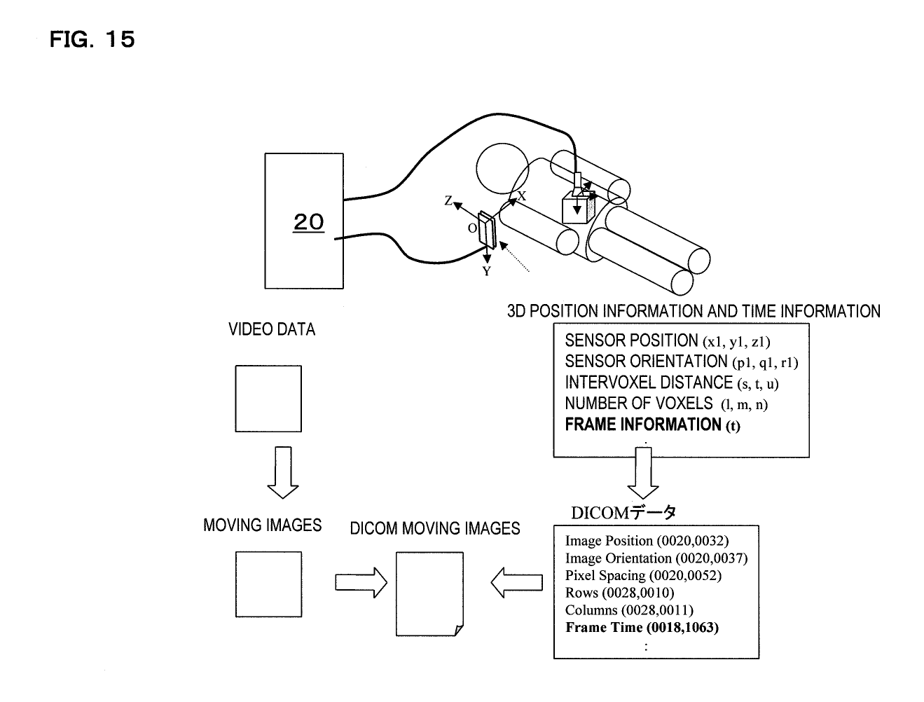

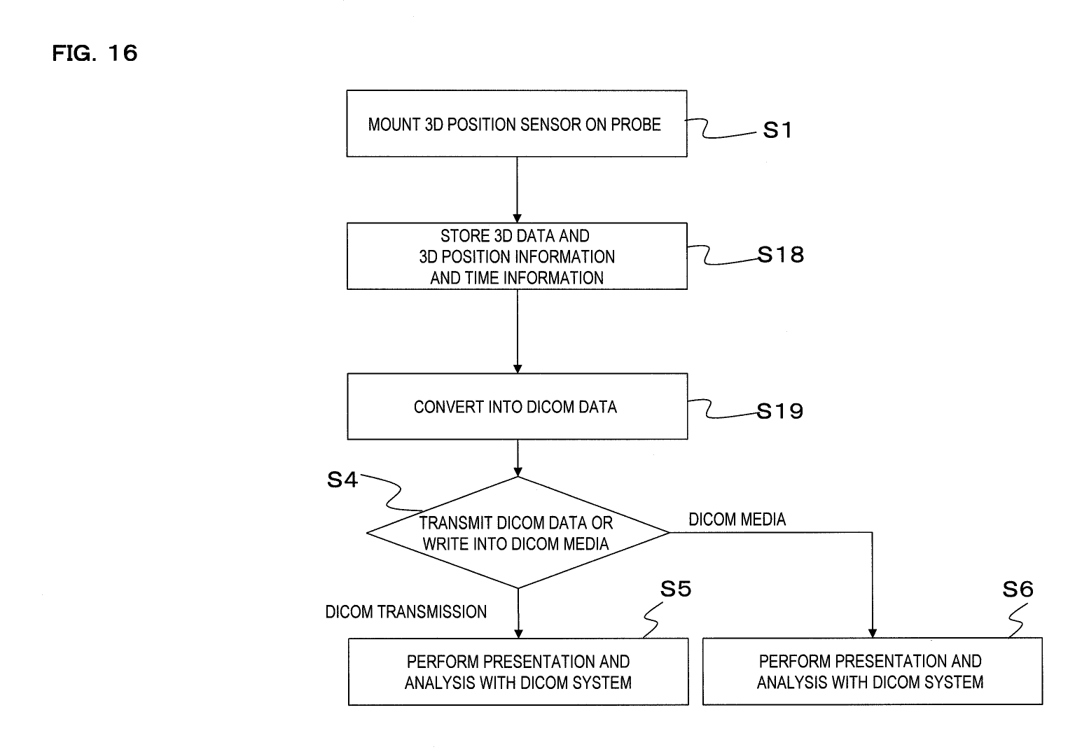

FIG. 15 shows a conceptual configuration diagram of the sixth embodiment of the ultrasound diagnostic system of the invention, and FIG. 16 shows a flowchart of a processing procedure of the present embodiment. The present embodiment is different from the other embodiments in that moving images are acquired solely by an ultrasound diagnostic system using the standard image data structure according to the invention to realize effective use.

As shown in FIG. 16, moving image data acquired by the ultrasound probe 1, the time information of the moving image data, the position and inclination of the position sensor detected by the 3D position detection means are acquired and stored (S18). Moreover, based on the time information and the detected position and inclination information of the position sensor, the time information, image position information, and inclination information of a predetermined DICOM data structure are set to the respective still image data of the stored moving image data. Then, the time information, image position information, and inclination information set to the data elements of the DICOM data structure are added to the respective still image data to generate DICOM video data (S19). In the DICOM data, as shown in FIG. 15, a Frame Time (0018, 1063) is defined in the data elements. The other aspects are the same as those of the first embodiment, and description thereof will be omitted.

According to the present embodiment, when making a diagnosis of an area of which the shape is different between being in the resting state and being in a stressed state as in a circulatory system as well as the heart or blood vessels, the moving images in the resting state and the stressed state are acquired and stored, the change (motion) in the shape of each part of the diagnostic area is analyzed. In such a case, by generating the 3D standard image data of the diagnostic area using the standard image data structure of the invention and detecting the 3D position of an arbitrary cross-section, it is possible to easily make a diagnosis through comparison with the previous examinations.

For example, in stress analysis of the heart, the videos in the resting state and the stressed state, of a certain cross-section of the heart are stored, and the motion of the atria and ventricles is analyzed. In this way, the state of each part of the heart can be detected. By detecting the 3D positions of cross-sections, it is possible to perform comparison with the previous examinations.

That is, in stress analysis of the heart, it is necessary to acquire the videos of a certain cross-section, and by detecting the 3D positions of cross-sections, it is possible to perform comparison with the previous examinations.

In the present embodiment, ultrasound video data may have any format such as JPEG. Examples of the time information of the DICOM data include frame information. The DICOM data conversion section 6 sets DICOM data elements including the 3D position information and the time information to moving images. As the time information, a Frame Time (0018, 106) is set, for example. The method of usage of the ultrasound DICOM image including the 3D position information and time information generated in such a way is the same as that of the first embodiment. Particularly, video presentation and video analysis of ultrasound DICOM images are performed by the destination DICOM server or the DICOM system which reads the DICOM files through media. The video presentation includes presentation through comparison on the same slice video. The video analysis includes 2D measurement of Doppler frequencies, elasticity, and the like. Moreover, the ultrasound diagnostic system 20 may read ultrasound DICOM images and perform video presentation and video analysis on the ultrasound DICOM images.

Preferred embodiments of the ultrasound diagnostic system and the like according to the invention have been described with reference to the accompanying drawings. However, the invention is not limited to the embodiments. It is clear that a person with ordinary skill in the art can easily conceive various modifications and changes within the technical idea disclosed herein, and it is contemplated that such modifications and changes naturally fall within the technical scope of the invention.

### REFERENCE SIGNS LIST

1: ULTRASOUND PROBE
2: ULTRASOUND TRANSMITTING AND RECEIVING CIRCUIT
3: ULTRASOUND SIGNAL CONVERSION SECTION
4: IMAGE DISPLAY SECTION
5: IMAGE AND IMAGE INFORMATION STORAGE SECTION
6: DICOM DATA CONVERSION SECTION
7: IMAGE TRANSMITTING SECTION
8: MEDIA R/W SECTION
9: POSITION SENSOR UNIT
10: POSITION INFORMATION INPUT SECTION

1. An ultrasound diagnostic system comprising:
an ultrasound probe configured to transmit and receive an ultrasound wave to and from a subject;
3D position detection means configured to detect the position and inclination of a position sensor with respect to the subject by the position sensor being mounted on the ultrasound probe;
storage means configured to acquire and store 3D image data acquired by the ultrasound probe scanning on the body surface of the subject and the position and inclination of the position sensor detected by the 3D position detection means;
standard image data setting means configured to divide the 3D image data stored in the storage means into a plurality of slice image data and set image position information and inclination information of a predetermined standard image data structure to the respective slice image data based on the position and inclination information of the position sensor detected by the 3D position detection means; and
standard image data generation means configured to generate 3D standard image data by adding the image position information and inclination information set by the standard image data setting means to the respective slice image data.

2. The ultrasound diagnostic system according to claim 1, wherein the standard image data structure further includes a pixel spacing of the respective slice image data and the respective numbers of pixel rows and columns, and
wherein the standard image data setting means calculates the distance between voxels and the number of voxels based on the 3D image data to set the pixel spacing and the respective numbers of the pixel rows and columns of the standard image data structure of the respective slice image data.

3. The ultrasound diagnostic system according to claim 1, further comprising:
coordinate conversion means that positions the position sensor on an anatomically distinct portion of the subject to adjust the position of origin of a position sensor coordinate system to the position of origin of a subject coordinate system.

4. The ultrasound diagnostic system according to claim 3, wherein the anatomically distinct portion is at least one of xiphisternum, subcostal processes, and hucklebone.

5. The ultrasound diagnostic system according to claim 1, wherein a 2D standard image in 3D standard image data captured by other modality imaging system are displayed on a monitor as a reference image, an ultrasound image acquired by the ultrasound probe while adjusting the position and inclination of the position sensor are displayed on the monitor, and the reference image and the ultrasound image are compared on the monitor to adjust a coordinate system of the position sensor to a subject coordinate system of the reference image so that the two images are made identical to each other.

6. The ultrasound diagnostic system according to claim 1, further comprising:
body motion detection means that detects at least one body motion waveform of an electrocardiogram waveform and a respiratory waveform,
wherein the storage means stores time information corresponding to characteristic points of a body motion waveform detected by the body motion detection means while acquiring the 3D image data,
wherein the standard image data structure includes the time information of the body motion waveform, and
wherein the standard image data setting means sets the time information to the standard image data structure of the respective slice image data.

7. The ultrasound diagnostic system according to claim 1, further comprising:
the storage means being configured to store 3D image data acquired by the ultrasound probe scanning in a direction perpendicular to a slicing cross-section of the subject at a constant speed and generate and store the 3D position and inclination information of the ultrasound probe based on the scanning of the ultrasound probe

8. An ultrasound diagnostic system comprising:
an ultrasound probe configured to transmit and receive an ultrasound wave to and from a subject;
3D position detection means configured to detect the position and inclination of a position sensor with respect to the subject by the position sensor being mounted on the ultrasound probe;
storage means configured to acquire and store moving image data acquired by the ultrasound probe, time information of the moving image data, and the position and inclination of the position sensor detected by the 3D position detection means;
standard image data setting means configured to set time information, image position information, and inclination information of a predetermined standard image data structure to the respective still image data of the moving image data stored in the storage means based on the time information and the position and inclination information of the position sensor detected by the 3D position detection means; and
standard image data generation means configured to generate video standard image data by adding the time information, image position information, and inclination information set by the standard image data setting means to the respective still image data.

9. A method for generating standard image data for an ultrasound diagnostic system, comprising:
a step wherein an ultrasound probe transmits and receives an ultrasound wave to and from a subject;
a step wherein 3D position detection means detects the position and inclination of a position sensor with respect to the subject by the position sensor being mounted on the ultrasound probe;
a step wherein a storage means acquires and stores 3D image data acquired by the ultrasound probe scanning on the body surface of the subject and the position and inclination of the position sensor detected by the 3D position detection means;
a step wherein standard image data setting means divides the 3D image data stored in the storage means into a plurality of slice image data and sets image position information and inclination information of a predetermined standard image data structure to the respective slice image data based on the position and inclination information of the position sensor detected by the 3D position detection means; and
a step wherein standard image data generation means generates 3D standard image data by adding the image position information and inclination information set by the standard image data setting means to the respective slice image data.

10. The method for generating standard image data for an ultrasound diagnostic according to claim 9, further comprising:
a step wherein the storage means stores 3D image data acquired by the ultrasound probe scanning in a direction perpendicular to a slicing cross-section of the subject at a constant speed and generates and stores the 3D position and inclination information of the ultrasound probe based on the scanning of the ultrasound probe

11. The method for generating standard image data for an ultrasound diagnostic according to claim 9, further comprising:
a step wherein the storage means acquires and stores moving image data acquired by the ultrasound probe, time information of the moving image data, and the position and inclination of the position sensor detected by the 3D position detection means;
a step wherein the standard image data setting means sets time information, image position information, and inclination information of a predetermined standard image data structure to the respective still image data of the moving image data stored in the storage means based on the time information and the position and inclination information of the position sensor detected by the 3D position detection means; and
a step wherein the standard image data generation means generates video standard image data by adding the time information, image position information, and inclination information set by the standard image data setting means to the respective still image data.
